(19) United States
(12) Patent Application Publication
(10) Pub. No.: US 2003/0013094 A1
(43) Pub. Date: Jan. 16, 2003

(54) Hybrid nucleic acid assembly

(76) Inventors: Michael L. Weiner, Webster, NY (US); Patrick R. Connelly, Rochester, NY (US); Carol Gebert, Cambridge (AU); Robert W. Gray, Rochester, NY (US)

Correspondence Address: GREENWALD & BASCH, LLP, 349 WEST COMMERCIAL STREET, SUITE 2490, EAST ROCHESTER, NY, 14445, US

(21) Appl. No.: 09/958,652

(22) PCT Filed: Feb. 17, 2001
(86) PCT No.: PCT/US01/05139

(51) Int. Cl. C12Q 1/68; C12M 1/34

(52) U.S. Cl. 435/6; 435/287.2

(57) ABSTRACT

A detection device containing of a hybrid nucleic acid assembly. The device contains a nucleic acid polymer, a first complementary oligonucleotide annealed to a first consensus sequence on the nucleic acid polymer, a second complementary ogligonucleotide annealed to a second consensus sequence on the nucleic acid polymer, a first nanoparticle conjugated with the first complementary oligonucleotide, a second nanoparticle conjugated with the second complementary oligonucleotide, means for introducing energy into the first nanoparticle, means for withdrawing energy from the second nanoparticle, means for detecting the withdrawal of energy from the second nanoparticle, and means for determining a physical property of the nucleic acid polymer while energy is introduced into the first nanoparticle.

### FIELD OF THE INVENTION

An assembly comprising a strand of nucleic acids joined to a signal transceiver, such as an electrically conductive carbon nanotube device.

### BACKGROUND OF THE INVENTION

In an article by Hans-Werner Fink and Christian Schonenberger, published in Nature (Volume 398, Apr. 1, 1999, at pages 407-410), the authors stated that: “The question of whether DNA is able to transport electrons has attracted much interest . . . . Experiments addressing DNA conductivity have involved a large number of DNA strands doped with intercalated donor and acceptor molecules, and the conductivity has been assessed from electron transfer rates as a function of the distance between the donor and acceptor sites. But the experimental results remain contradictory, as do theoretical predictions.”

The prior art techniques for measuring DNA conductivity are relatively crude; with many of such techniques, the acts of measurement influence the very variables being measured.

Additionally, to the best of applicants' knowledge, none of the prior studies of DNA conductivity measured such conductivity with the DNA in an environment similar to its natural environment.

It is an object of this invention to provide a process for measuring DNA conductivity which is substantially more accurate than prior art processes.

It is another object of this invention to provide a process for measuring DNA conductivity (of electrons, photons, and vibration) while such DNA is undergoing its normal processes (such as transcription or replication) in substantially its normal environment.

It is another object of this invention to provide a process for measuring the shape structure of DNA.

It is another object of this invention to provide a novel hybrid nucleic acid assembly useful in practicing the processes of this invention.

### SUMMARY OF THE INVENTION

In accordance with this invention, there is provided a hybrid nucleic acid assembly comprised of a partially denatured double strand of nucleic acid, a first probe attached to a proximal end of such strand, and a second probe attached to a distal end. Each of the first and second probes is comprised of a conductive fiber.

### BRIEF DESCRIPTION OF THE DRAWINGS

The present invention will be described by reference to the following drawings, in which like numerals refer to like elements, and in which:

FIG. 1 is a flow diagram of one preferred process of the invention;

FIG. 1A is a flow diagram of a process for making certain transceivers;

FIGS. 2A, 2B, and 2C are schematic representations of various transceiver assemblies which may be used in the process of the invention;

FIGS. 3A and 3B are schematic diagrams of a measurement device utilizing the hybrid nucleic acid assembly of FIG. 2; and

FIG. 4 is a flow diagram of an imaging process for detecting the shape structure of DNA.

### DESCRIPTION OF THE PREFERRED EMBODIMENTS

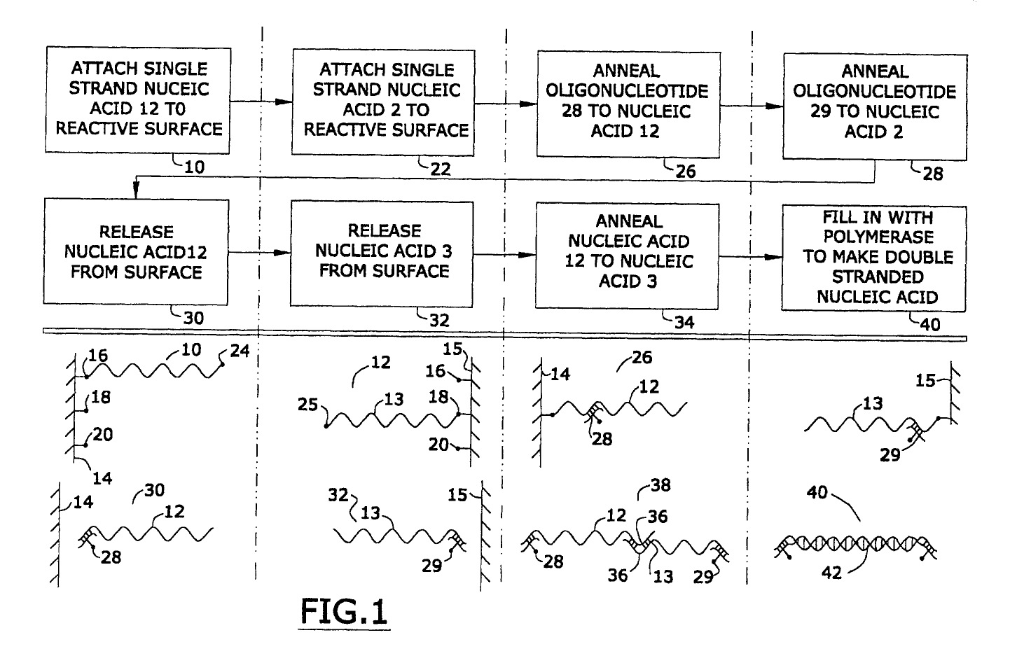

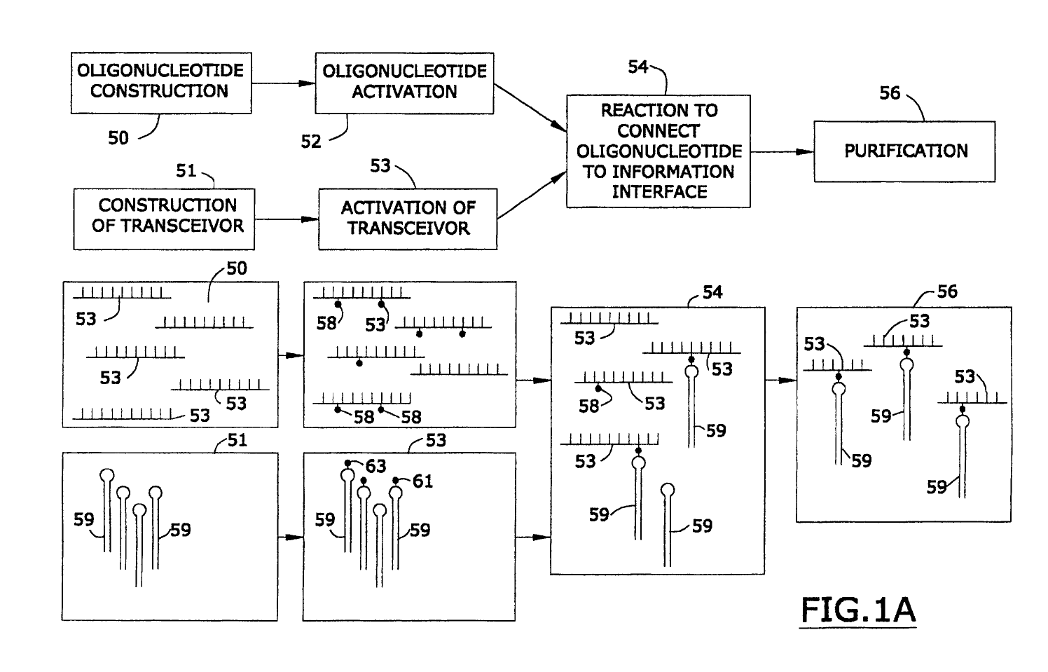

FIG. 1 is a flow diagram of one preferred process of the invention. FIG. 1A is a schematic diagram illustrating how preferred oligonucleotide assemblies used in the process of FIG. 1 may be assembled.

Referring to FIG. 1, and in the preferred embodiment depicted therein, in step 10 of the process, a single strand of nucleic acid 12 is attached to a reactive surface 14 comprised of reactive sites 16, 18, and 20.

One may prepare single stranded DNA by conventional means, such as, e.g., cDNA preparation techniques; see, e.g., U.S. Pat. Nos. 6,184,017, 6,180,612, 6,180,385, 6,177,244, and 6,172,197, the entire disclosure of each of which is hereby incorporated by reference into this specification.

The single stranded DNA used in the process depicted in FIG. 1 is the sequence to be analyzed. It may, e.g., contain promoter and/or enhancer regions, structural genes (including introns, exons, and the like), etc.

It is preferred that the single stranded DNA 12 be purified, i.e., it be substantially homogeneous. DNA purification may be effected by conventional means; see, e.g., U.S. Pat. Nos. 6,187,578, 6,187,575, 6,187,564, 6,187,559, 6,187,552, and the like, the entire disclosure of each of which is hereby incorporated by reference into this specification.

Reactive surface 14 may be any surface commonly used in the preparation of DNA chips. Such DNA chips are well known and are sold, e.g., by the Affymetrix Company, by the Incyte Company, etc. Alternatively, the reactive surface 14 may be a passive derivatized polymeric or glass surface. The preparation of derivatized reactive surfaces is well known; see, e.g., U.S. Pat. Nos. 5,641,539, 5,453,199, 5,372,719, and the like, the entire disclosures of each of which is hereby incorporated by reference into this specification.

In one embodiment, the reactive surface 14 is described in an article by Brett A. Stilman et al. Entitled “FAST Slides: A Novel Surface for Microarrays” appearing in the September, 2000 edition (Volume 29, No. 3) of BioTechiques, at pages 630-635.

Referring again to FIG. 1, one may attach single stranded DNA 12 to reactive surface 14 by conventional techniques. Thus, by way of illustration and not limitation, one may attach strand 12 to surface 14 by covalent bonding. See, e.g., U.S. Pat. Nos. 5,472,888 and 6,177,247, the entire disclosure of each of which is hereby incorporated by reference into this specification.

In step 22, another strand of preferably purified DNA 13 is attached to reactive surface 15, which preferably has characteristics similar to surface 14 but may differ therefrom. It is preferred that DNA strand 13 have a base sequence that differs from DNA strand 12. As will be apparent to those skilled in the art, DNA strand 13 also is derived from the DNA to be analyzed.

In another embodiment, two different reactive surfaces are used to bond to DNA strands 12 and 13, respectively.

In one embodiment, strands 12 and 13 have similar base sequences. What is required however, in all embodiments, is that end 24 of strand 10 and end 25 of strand 13 have complementary base pairs to facilitate annealing therebetween.

In one preferred embodiment, depicted in FIG. 1, each of steps 10 and 22 occur in different environments, such as, e.g., separate test tubes. In this embodiment, two distinct, noncontiguous reactive surfaces are used.

In step 26 of the process, an oligonucleotide 28 is annealed to DNA strand 12 by conventional means; see, e.g., U.S. Pat. Nos. 6,083,723, 6,083,698, 6,051,379, 6,017,731, 5,972,604, and the like, the entire disclosure of each of which is hereby incorporated by reference into this specification. As will discussed in detail elsewhere in this specification, oligonucleotide 28 (and oligonucleotide 29) is attached to a device, which is capable of generating an electrical or magnetic or optical signal or otherwise transmitting information.

It is preferred that oligonucleotide 28 contains base pairs complementary to the base pairs in the region of DNA strand 12 to which oligonucleotide 28 is to be annealed. Similarly, oligonucleotide 29 preferably contains base pairs complementary to a region of DNA strand 13; and in step 28 oligonucleotide 29 is similarly annealed to DNA strand 13.

In steps 30 and 32, the annealed DNA strands 12 and 13 are optionally caused to be released from reactive surfaces 14 and 15, respectively. This is preferably done by the breaking of the chemical bond between strands 12/13 and surfaces 14/15. This breaking may be effected by conventional means such as, e.g., restriction enzyme cleavage.

In one embodiment, step 30 and/or step 32 is omitted. In one embodiment, only one of strands 12 or 13 is released. Thus, e.g., one may have a situation in which the bond energy between strand 12 and surface 14 is substantially greater than the bond energy between strand 13 and surface 15, in which case the latter bond is preferentially broken after strands 12 and 13 are hybridized. By way of illustration, surfaces 14 and 15 may exist on a flat or spherical surface (such as, e.g., beads).

One may cleave the DNA strands 12/13 by conventional means such as,e.g., those disclosed in U.S. Pat. Nos. 6,183,993, 6,180,402, 6,180,338, 6,175,001, 6,174,724, and the like; the entire disclosure of each of these United States patents is hereby incorporated by reference into this specification.

The released strands 12/13 which are bonded to oligonucleotides 28/29 are then preferably pooled by charging them to the same container, which preferably contains a buffer, such as tris-buffer. Thereafter, in step 34, the released strands 12/13 anneal to each other in mutually complementary region.

The annealed structure 38 is partially single stranded and partially double stranded. In step 40, annealed structure 38 is made completely double stranded by exposing it to base building blocks (nucleotides) in the presence of polymerase and ligase, in accordance with standard protocols for DNA vector construction. See, e.g., U.S. Pat. Nos. 4,853,323, 6,184,034, 6,184,000, 6,177,543, 6,171,861, and the like.

In the embodiments depicted in FIG. 1, oligonucleotides are depicted as being disposed on opposite strands 12 and 13. In another embodiment, not shown, the oligonucelotides are disposed on the same strands. In yet another embodiment, not shown, the oligonucleotides are disposed on both the same and on opposite strands. Other combinations will be apparent to those skilled in the art.

Referring again to FIG. 1, the double-stranded structure 42 produced by this process has incorporated within it oligonucleotides 28 and 29.

FIG. 1A illustrates one preferred process for constructing oligonucloetide devices containing oligonucleotide 28 or 29 and, attached to each such entity, an information interface. In step 50 of this process, the desired oligonucleotide 53 with the desired base sequence(s) is constructed by conventional means and/or purchased.

In step 52 of the process, the oligonucleotide is activated at sites 55 and 57; as will be apparent to those skilled in the art, the oligonucleotide activation step is variable. Some of the oligonucleotides are not activated, some are activated at one site, and some or activated at two or more sites.

The term oligonucleotide activation, as used in this specification, refers to chemical modification of the nucleic acid(s) within the oligonucleotide to enable such nucleic acid(s) to react with another molecule located on a transceiver. One may activate the oligonucleotide by conventional means. Thus, e.g., one may use alkylthio functionalization activation of DNA; see, e.g., an article by Gregory P. Mitchell et al. Entilted “Programmed Assembly of DNA Functionalized Quantum Dots” (Journal of the American Chemical Society, 1999, 121, pages 81232-8123). Thus, one may use methylation of DNA. Thus, e.g., one may use the techniques described in an article by Jens-Peter Knemeyer et al. Entitled “Probes for Detection of Specific DNA Sequences . . . ,” appearing in Analytical Chemistry, Volume 72, pages 3717 to 3724 (Aug. 15, 2000). Thus, e.g., one may use one or more of the activation techniques described in U.S. Pat. Nos. 6,013,789, 5,577,694, 5,747,244, 5,712,383, 5,612,468, 5,525,711, and the like; the entire disclosure of each of these United States patents is hereby incorporated by reference into this specification.

A suitable transceivor 59, as defined below, is constructed in step 51, and the transcivor 59 is activated in step 53 at sites 61 and 63.

In a similar manner, the surface of the transceiver may be activated in transceiver activation step 53. As used herein, the term transceivor refers to any molecule, compound, structure, or article capable of either conducting a signal, transducing a signal from one form to another, initiating a signal, etc.

By way of illustration, the transceiver may be a metal fiber such as, e.g., a gold fiber. By way of further illustration, the transceivor may be a carbon nanotube fiber, a chromophor (which changes its optical properties upon excitaton), a fluorophor (which also changes its optical properties upon excitation), a lumiphor (which emits photons upon receipt of electrons), a molecular battery (which releases electricity upon photon stimulation), a radio frequency antenna (which receives or transmits radio frequency energy upon excitation), and the like. Additionally, the transceivor may be comprised of a radioactive material whose decay initiates one or more chemical reactions, resulting in the discharge of electrons.

Regardless of the structure of the transceivor, or its form or composition, it is preferred to activate such transceiver in step 53 so that it will readily bond to the activated oliogonucelotide, preferably by chemical means. The bonding may occur by any known mechanism such as, e.g., by Van Der Waals forces, by ionic forces, by polar forces, by combinations thereof, and the like. In one preferred embodiment, the bonding is effected primarily by covalent bonds.

Referring again to FIG. 1A, and in the preferred embodiment depicted therein, in step 54 the activated oligonucleotide is reacted with the activated transceiver to produce an assembly adapted to receive information from or transmit information to a DNA sequence.

In step 56, the reaction products produced in step 54 may be purified by conventional means such as, e.g., chromatography to insure that the reaction products are substantially homogeneous. See, e.g., U.S. Pat. Nos. 6,187,578, 6,187,585, 6,187,564, 6,187,559, and 6,187, 552; the entire disclosure of each of these United States patents is hereby incorporated by reference into this specification.

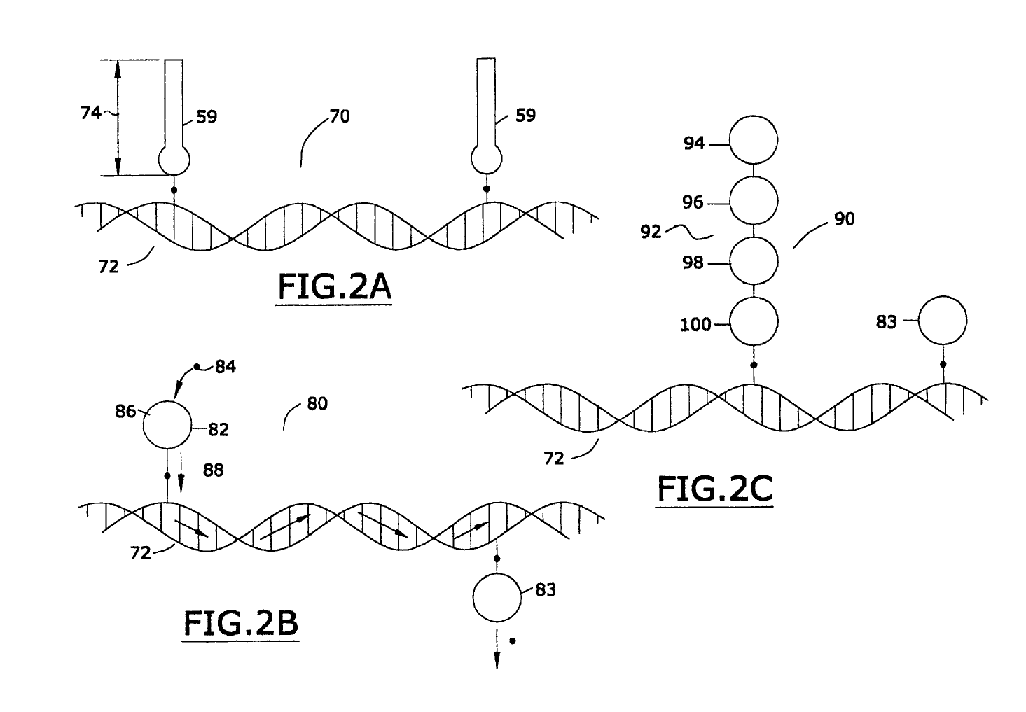

FIG. 2A is a representation of a transceiver assembly 70 comprised of a double stranded DNA 72 produced in accordance with steps 28 and 40 of the process depicted in FIG. 1. In the embodiment depicted in FIG. 2A, the transceivers 59 are carbon nanotubes which have a length 74 such that they preferably extend out of the environment in which the double stranded DNA 72 is disposed. Thus, for example, if the DNA 72 is disposed within a test tube (not shown), the tops 76 of transceivers 59 preferably extend beyond the top of the test tube. As will be apparent, this feature enables other devices to be readily attached to the tops 76 of the nanotubes 59; and it also facilitates the use of such other devices whose use might be interefered with given the physical constraints of the test tube.

In the embodiment 70 depicted in FIG. 2A, each of the transceivers 59 is a carbon nanotube. In the transceiver assembly 80 depicted in FIG. 2B, each of the transceivers 82 and 83 is a molecule which, preferably, is a fluorophore (a potentially fluorescent group of atoms in a molecule) which will fluoresce upon being excited; see, e.g., U.S. Pat. Nos. 6,187,567, 6,187,566, 6,187,250, 6,184,027, 6,183,984, and the like, the entire dislcosure of each of which is hereby incorporated by reference into this specification. Transceivors 82 and 83 may consist essentially of the same material, or they may consist essentially of different materials.

If a photon 84 impinges upon the surface 86 of fluorophoric transceivor 82, the transceiver 82 will emit an electron (not shown) which will travel into the double stranded DNA 72 in the direction of arrow 88 and thence through the DNA 72 in the direction of arrows 90 until it is received by receptor transceivor 82′, which attracts it because of a potential difference. When the electron is received by trasceivor 82′, it will emit a photon, fluoresce, and then return to its base state potential which existed before it received the electron. As will be apparent, this device enables the construction of more complicated circuits.

The embodiment depicted in FIG. 2C is similar to that depicted in FIG. 2B with the exception that the transceiver 92 is comprised of a multiplicity of fluorophoric sites 94, 96, 98, and 100. When such a transceiver 92 is excited by a multiplicity of electrons (not shown), it will emit substantially more electrons than the single fluorophore depicted in FIG. 2B; and it will thus cause the transceiver 83 to fluoresce substantially longer and/or more frequently.

As will be apparent, a photon can cause the emission of an electron in a fluorphoric device. It will be apparent, however, that this process is reversible, and that an electron can cause the emission of a photon from a fluorophoric device.

One can determine, under normal conditions for a specified double stranded DNA, how much a specified amount of excitation energy causes a current to flow, especially if measuring devices are connected to the transceivers. Consequently, one can determine when there is any aberrant condition with such DNA that would affect such current flow, and/or one can determine when normal DNA processes (such as transcription or replication) are occurring. Reference data can be generated as to the current flows normally existing during these events, and such data can be correlated with readings taken from the DNA when it is in a substantially in vivo environment.

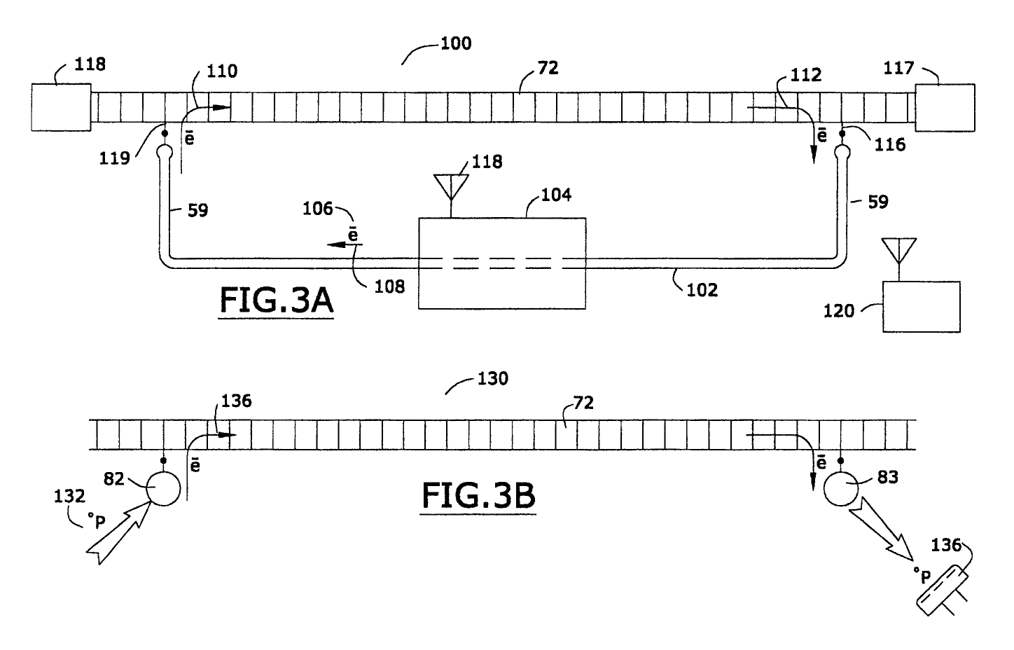

FIG. 3A is a schematic representation of a circuit 100 in which transceivers 59 are conductively connected by conductor 102 to form a closed circuit comprised of such transceivers 59, such conductor 102, and double stranded DNA 72. Disposed within such circuit 100 is controller 104 which is capable of sending energy 106 around the circuit 100 in the directions of arrows 108, 110, and 112. The energy transmitted by controller 104 may be thermal energy, and/or light energy, and/or vibrational energy, and/or magnetic energy, and/or electrical energy. Any of the forms of such energy commonly available or producible may be sent by controller 104.

By way of illustration and not limitation, the energy transmitted may be electrical energy that is either direct current energy or alternating current energy. When alternating current energy is sent, it may be amplitude modulated energy, frequency modulated energy, phase modulated energy, and the like. As will be apparent, one may vary the amplitude, voltage, frequency, current, and impedance of such energy, as is well known in the electrical art.

By way of further illustration., the energy transmitted may be light energy, either in the form of waves and/or particles, at various frequencies, wavelengths, or combinations thereof. In this embodiment, the conductor 102 may be a fiber optic conductor.

The controller 104, in addition to emitting energy, also is capable of measuring the characteristics of the DNA between points 114 and 116. As will be apparent, although only two connection points 114 and 116 are depicted in FIG. 3A, more of such connections could be made so that one could determine the electrical characteristics between any two points on DNA strand 72.

The electrical properties of DNA strand 72 will vary depending upon its geometry and chemical composition. These characteristics will, in turn, vary when events such as protein binding, transcription, replication, denaturation, and the like occur. Thus, the circuit 100 may be used to determine when a particular strand 72 of DNA is undergoing such an event and/or whether a particular strand of DNA 72 evidences an aberrant behavior or composition or geometry which affects such electrical characteristics.

The controller 104 is preferably comprised of a programmable logic chip which enables it to modify its performance upon evaluation of the data it collects from DNA strand 72.

In one embodiment, the circuit 100 is disposed in an environment (not shown) which substantially simulates and/or is substantially identical to the environment the DNA strand 72 normally is in. Thus, one may charge circuit 100 to an aqueous environment comprised of buffer, essential biological components (such as nucleotides, adenosine triphosphate, protein enzymes), and the like. Thus, one may ligate circuit 100 into a DNA vector (not shown) by means of linkers 115 and 117, in accordance with standard biotechnical protocols; see, e.g., U.S. Pat. Nos. 6,187,757, 6,183,753, 6,180,782, 6,136,568, 6,136,318, and the like, the entire disclosure of each of which is hereby incorporated by reference into this specification. Thus, the circuit 100 may be introduced into a functional biological entity such as a bacteria or eukaryotic cell and, thereafter, used to monitor the in vivo activity within the bacterium or eukaryotic cell. As will be apparent, the vector containing circuit 100 can be made part of any nucleic entity (such as, e.g., a plasmid, a chromosome) and, after such modification, undergoes precisely the same occurrences as would an endogenous species. Thus, the circuit 100 may be used to evaluate and monitor and modify a wide variety of in vivo activities.

When the circuit 100 is disposed within a cell, e.g., and when the oligonucleotides 28 and 29 are disposed on opposite strands 12 and 13, when such a cell divides, each daughter cell will then receive one end of the circuit 100, in which case the circuit 100 measures conductivity across two or more cells.

In another embodiment, not shown, the circuit 100 is encapsulated in a lipid derived delivery system prior to being incorporated within a cell. This technique is well known and is described, e.g., in U.S. Pat. No. 6,187,760, the entire disclosure of which is hereby incorporated by reference into this specification. As is disclosed in such patent, the introduction of foreign nucleic acids and other molecules is a valuable method for manipulating cells and has great potential both in molecular biology and in clinical medicine. Many methods have been used for insertion of endogenous nucleic acids into eukaryotic cells. E.g., see Graham and Van der Eb, Virology 52, 456 (1973) (co-precipitation of DNA with calcium phosphate); Kawai and Nishizawa, Mol. Cell. Biol. 4, 1172 (1984) (polycation and DM80); Neumann et al., EMBO Journal 1, 841 (1982) (electroporation); Graessmann and Graessmann in Microinjection and Organelle Transplantation Techniques, pp. 3-13 (Cells et al., Eds., Academic Press 1986) (microinjection); Cudd and Nicolau in Liposome Technology, pp. 207-221 (G. Gregoriadis, Ed., CRC Press 1984) (liposomes); Cepko et al., Cell 37, 1053 (1984) (retroviruses); and Schaffher, Proc. Natl. Acad. Sci. USA 77, 2163 (1980) (protoplast fusion). Both transient and stable transfection of genes has been demonstrated.

Some of the first work on liposome delivery of endogenous materials to cells occurred about twenty years ago. Foreign nucleic acids were introduced into cells (Magee et al., Biochim. Biophys. Acta 451, 610-618 (1976), Straub et al., Infect. Immun. 10, 783-792 (1974)), as were foreign lipids (Martin and MacDonald, J. Cell Biol. 70, 5 15-526 (1976)), Proteins (Magee et al., J. Cell. Biol. 63, 492 (1974), Steger and Desnick, Biochim. Biophys. Acta 464, 530 (1977)), fluorescent dyes (Leventis and Silvius), and drugs (Juliano and Stamp, Biochem. Pharm. 27, 2127 (1978), Mayhew et al., Cancer Res. 36, 4406 (1976), Kimelberg, Biochim. Biophys. Acta 448, 531 (1976)), all using positively charged lipids.

Of the many methods used to facilitate entry of DNA into eukaryotic cells, cationic liposomes are among the most efficacious and have found extensive use as DNA carriers in transfection experiments. See, generally, Thierry et al. in “Gene Regulation: Biology of Antisense RNA and DNA,” page 147 (Erickson and Izant, Eds., Raven Press, New York, 1992); Hug and Sleight, Biochim. Biophys. Acta 1097, 1(1991); and Nicolau and Cudd, Crit. Rev. Ther. Drug Carr. Sys. 6, 239 (1989). The process of transfection using liposomes is called lipofection. Senior et al., Biochim. Biophys. Acta 1070, 173 (1991), suggested that incorporation of cationic lipids in liposomes is advantageous because it increases the amount of negatively charged molecules that can be associated with the liposome. In their study of the interaction between positively charged liposomes and blood, they concluded that harmful side effects associated with macroscopic liposomeplasma aggregation can be avoided in humans by limiting the dosage.

Feigner et al., Proc. Natl. Acad. Sci. USA 84, 7413 (1987), demonstrated that liposomes of dioleoylphosphatidylethanolamine (DOPE) and the synthetic cationic lipid N-[1-(2,3-dioleyloxy)propyl]-N,N,N-trimethylammonium chloride (DOTI VIA) are capable of both transiently and stably transfecting DNA. Rose et al., BioTechiques 10, 520 (1991), tested lipofection with liposomes consisting of DOPE and one of the cationic lipids cetyldimethylethylammonium bromide (CDAB), cetyltrimethylethylammonium bromide (CTAB), dimethyldioctadecylammonium bromide (DDAB), methylbenzethoniurn chloride (MBC) and stearylamine. All of the liposomes (except that with CTAB) successfully transfected DNA into HeLa cells. At high concentrations; however, CDAB and MBC caused cell lysis. Only DDAB was found to be effective in mediating efficient DNA transfection into a variety of other cell lines. Malone et al., Proc. Natl. Acad. Sci. USA 86, 6077 (1989), successfully transfected RNA, in vitro, into a wide variety of cells lines. Zhou and Haung, J. Controlled Release 19, 269 (1992), disclosed successful lipofection by DOPE liposomes stabilized in the lamellar phase by cationic quaternary ammonium detergents. The authors noted, however, that the relatively high cytotoxicity of these compounds would limit their use in vivo.

Hawley-Nelson et al., Focus 15, 73 (1990, BRL publications), disclosed the cationic lipid “LIPOFECTAMH′4E”, a reagent containing 2,3-dioleyloxy-N-[2(spermineearboxyaniido)ethyl]-N,N-dimethyl-1-propanamin ium trifluoroacetate (DOSPA). “LEPOFECTAMJNE” was found to have higher transfection activity than several monocationic lipid compounds (“LIPOFECTIN”, “LIPOFECTACE”, and DOTAP) in six of eight cell types tested. They observed toxicity when both lipid and DNA were included in the same mixture. These encapsulating agents are sold by Gibco BRL Systems, Inc. of Bethesda, Md.

Referring again to FIG. 3A, and in the embodiment depicted therein, an antenna 118 is operatively connected to the controller 104 and is adapted to transmit signals in response to instructions from such controller. In one aspect of this embodiment, the antenna 118 emits signals in response to readings taken of the DNA strand 72. Inasmuch as the electrical properties of DNA strand 72 vary substantially instantaneously when various biochemical events occur, a remote receiver 120 disposed outside of circuit 100 may be used to monitor the status of and the activity of such DNA. This is especially useful when circuit 100 is disposed within a living being.

FIG. 3B depicts a circuit 130 similar to circuit 100 but differing therefrom in that the energy 132 is introduced from a source remote from circuit 130. One may use any conventional remote energy source such as, e.g., a laser, sound, radio frequency energy, magnetism, and the like. In the embodiment depicted, upon the introduction of photonic energy 132, an electron 134 is caused to flow within the DNA strand 72 and flows to the lower potential transceivor 83, which it causes to emit energy in the form of a photon and causes it to fluoresce. The fluorescence may be detected by a photodector 136 which, depending upon the intensity and frequency of the fluorescence, will monitor and measure activity within DNA strand 72.

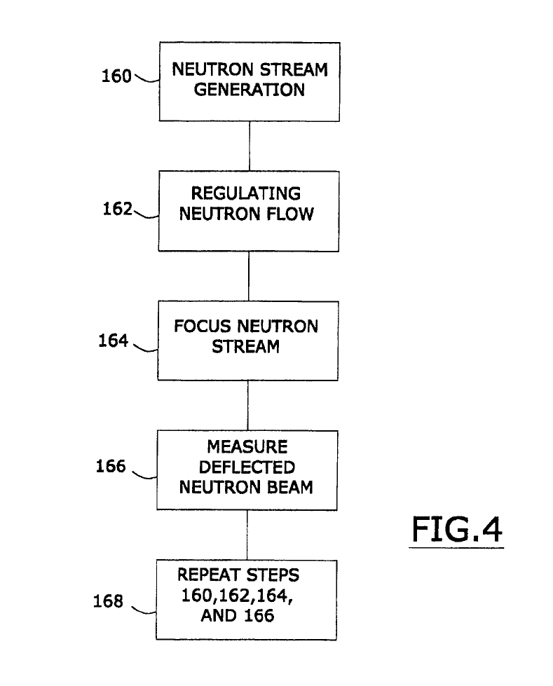

FIG. 4 is a flow diagram of a process for imaging chromosomal events in biological systems. In the first step of the process depicted in FIG. 4, in step 160, a neutron stream is produced by conventional means such as, e.g., a cyclotron. See, e.g., U.S. Pat. Nos. 5,699,394, 5,386,114, 4,853,550, 4,701,792, 4,176,093, and the like; the entire disclosure of each of these United States patents is hereby incorporated by reference into this specification.

Thereafter in 162 of the process, the flow of the neutron stream is regulated by means of, e.g., supercooled fluid. See, e.g., U.S. Pat. Nos. 5,872,826, 5,610,956, 5,367,547, 5,174,945, 5,128,097, and the like. The entire description of each of these United States patents is hereby incorporated by reference into this specification.

In step 164 of the process, the slowed neutron stream is focused onto a nucleus of a cell which one wishes to scan. See, e.g., U.S. Pat. Nos. 6,054,708, 5,658,273, 5,076,993, etc., the entire disclosure of each of which is hereby incorporated by reference in to this specification. In this step 164, the cell nucleus is bombarded with the slowed neutron stream, which is preferably focused onto desired target chromosomes.

In one embodiment, streams of neutrons are simultaneously focused on different portions of the nucleus to produce a three-dimensional image. In this embodiment, one may scan the beams over the portions of the nuclei being examined to produce a real time image.

The neutrons impacting the cell nuclei are transmitted and deflected. In step 166, these deflected neutron beams are measured. The extent of the transmission and deflection of the neutron beam, and the intensity of each, varies with the mass contacted by the neutron beam. Thus, one can determine deflection patters of neutron beams for standardized DNA samples and compare this data with the deflection patterns produced with any particular DNA sample. This data correlation will indicate whether any particular DNA is aberrant, and/or is undergoing an event such as replication or transcription.

The transmitted and/or deflected neutron beams are anaylzed in a controller comprised of a neutron detector, a monitor, image processing software, so that the shape, structure, motion, configuration, and relative positioning of the chromosomal moieties within the nuclei may be viewed and evaluated. By way of illustration and not limitation, one may thus examine, evaluate, and quantify phenomena such as the positioning of the histones and nucleosomes within chromatin, the spatial relationship between chromosomes, looping of the DNA, psuedo knot formation within the DNA, supercoiling within the DNA, unwinding of the DNA, the energy state of the DNA, and any other structural property normally or abnormally exhibited by chromatin. The observation, evaluation, and compilation of these properties allow one to prepare a database of normal and aberrant states of the cell.

In step 168, steps 160, 162, 164, and 166 are repeated as the focus of the neutron beam is changed. Thus, one can scan a DNA segment in real time and instantaneously generate data indicate its condition and what events, if any, it is undergoing.

The steps 160 through 168 can be repeated with a wide variety of standardized DNA samples, and test DNA samples, to produce a database that can thereafter be correlated with any particular set of readings. By way of illustration, such a database will have data regarding the phenotypes of individuals with specific maladies, such as disease state, age, etc. When a scanning of any particular DNA sample matches such a malady phenotype, one then gains an indicium of the possibility of such malady existing or developing.

In practicing the techniques described in this specification, one may use procedures described in prior art patents. Thus, one should refer to U.S. Pat. No. 5,612,468 (Pteridine nuleotide analogs as fluorescent DNA probes),U.S. Pat. No. 5,846,708 (Optical and electrical methods and apparatus for molecule detection), U.S. Pat. Nos. 4,447,546, 4,582,809, 4,909,990, 5,776,672 (Gene detection niethod), U.S. Pat. No. 6,146,593 (High density array fabrication and readout method for fiber optic biosensor), U.S. Pat. No. 6,146,593, etc. The entire disclosure of each of these United States patents is hereby incorporated by reference into this specification.

As indicated elsewhere in this specification, the transceivors 59 (see FIG. 3A) are preferably nanotubes. As used this specification, the term nanotube includes a nanoparticle, which can be fabricated from gold, from carbon, from other materials, and may be fabricated in substantially any shape.

In one embodiment, the nanoparticle consists essentially of gold. In another embodiment, the nanoparticle is a nanotube which can contain a single wall, or a double wall, or a multiplicity of walls. These walled nanotubes are well known to those skilled in the art. See, e.g., U.S. Pat. Nos. 6,187,760, 6,187,823, 6,183,174, 6,159,742, and the like. The entire disclosure of each of these United States patents is hereby incorporated by reference into this specification.

The invention has been described by reference to deoxyribose nucleic acid structures, as well as ribonucleic acid structures. It is equally applicable to protein structures, to which an antibody may be bonded in conventional manner to different moieties on the protein entity.

It is to be understood that the aforementioned description is illustrative only and that changes can be made in the apparatus, in the ingredients and their proportions, and in the sequence of combinations and process steps, as well as in other aspects of the invention discussed herein, without departing from the scope of the invention as defined in the following claims.

We claim:

1. A detection device comprised of a hybrid nucleic acid assembly, wherein said hybrid nucleic acid assembly is comprised of a nucleic acid polymer comprised of a proximal end and a distal end, a first complementary oligonucleotide annealed to a first consensus sequence on said nucleic acid polymer, a second complementary ogligonucleotide annealed to a second consensus sequence on said nucleic acid polymer, a first nanoparticle conjugated with said first complementary oligonucleotide, a second nanoparticle conjugated with said second complementary oligonucleotide, means for introducing energy into said first nanoparticle, means for withdrawing energy from said second nanoparticle, means for detecting the withdrawal of energy from said second nanoparticle, and means for determining a physical property of said nucleic acid polymer while said energy is introduced into said first nanoparticle.

2. The detection device as recited in claim 1, wherein said nucleic acid polymer is a double stranded sequence of deoxyribonucleic acid.

3. The detection device as recited in claim 1, wherein said nucleic acid polymer is a single stranded sequence of deoxyribonucleic acid.

4. The detection device as recited in claim 1, wherein said first nanoparticle consists essentially of gold.

5. The detection device as recited in claim 4, wherein said second nanoparticle consists essentially of gold.

6. The detection device as recited in claim 1, wherein said first nanoparticle and said second nanoparticle is a nanotube.

7. The detection device as recited in claim 6, wherein said nanotube is a carbon nanotube.

8. The detection device as recited in claim 7, wherein said carbon nanotube is a singlewalled carbon nanotube.

9. The detection device as recited in claim 7, wherein said carbon nanotube is a doublewalled carbon nanotube.

10. The detection device as recited in claim 1, wherein said means of introducing energy into said first nanoparticle is disposed externally of said hybrid nucleic acid assembly.

11. The detection device as recited in claim 10, wherein said means of introducing energy into said first nanoparticle is a light source.

12. The detection device as recited in claim 10, wherein said means of introducing energy into said first nanoparticle is a laser.

13. The detection device as recited in claim 10, wherein said means of introducing energy into said first nanoparticle is a source of radio frequency energy.

14. The detection device as recited in claim 10, further comprising means for producing a radio frequency signal which contains information regarding said physical property of said nucleic acid polymer.

15. The detection device as recited in claim 1, wherein said means for detecting the withdrawal of energy from said second nanoparticle is photodetector.

16. The detection device as recited in claim 1, wherein said means for detecting the withdrawal of energy from said seocond nanopoarticle is a radio frequency receiver.

17. The detection device as recited in claim 1, wherein said detection device is a nanoparticle.

18. The detection device as recited in claim 1, wherein said detection device is incorporated into an encapsulating agent.

19. The detection device as recited in claim 18, wherein said detection device whereas said detection device is transfected into a cell.
